(19) United States
(12) Patent Application Publication
(10) Pub. No.: US 2007/0038094 A1
(43) Pub. Date: Feb. 15, 2007

(54) System and method for the creation of ultrasonic waves

(76) Inventor: Ilja Kruglikov, Linkenheim-Hochstetten (DE)

Correspondence Address: BRINKS HOFER GILSON & LIONE, P.O. BOX 10395, CHICAGO, IL, 60610, US

(21) Appl. No.: 11/490,837

(22) Filed: Jul. 21, 2006

(30) Foreign Application Priority Data
Jul. 27, 2005 (EP) EP 05016273.4
Jul. 18, 2006 (EP) EP 06014899.6

(51) Int. Cl. A61B 8/00

(52) U.S. Cl. 600/438

(57) ABSTRACT

A system for generating ultrasonic waves may be used for cosmetic and therapeutic treatments. The system includes an electrically controlled ultrasonic head that generates ultrasonic waves at multiple different frequencies. The system also includes a control unit that switches between the multiple frequencies. The system may switch the frequencies at a modulation frequency between 10 Hz and 10,000 Hz, between 50 Hz and 5,000 Hz, between 100 Hz and 2,000 Hz, or between other frequency ranges.

### BACKGROUND OF THE INVENTION

1. Priority Claim

This application claims the priority benefit of European patent application EP 05016273.4, filed 27 Jul. 2005, and European patent application EP 06014899.6, filed 18 Jul. 2006, both of which are entirely incorporated by reference.

2. Technical Field

The invention relates to a system for the generation and application of ultrasonic waves at multiple different frequencies for therapeutic purposes.

3. Related Art.

Ultrasonic waves are used to effect biological tissues for medical, therapeutic or beauty applications as well as for research purposes. Usually the acoustic vibrations with a frequency higher than 20 kilohertz are called “ultrasonic” waves. The term “low-frequency” ultrasonic is frequently used for the range of 20 to 100 kHz and the term “high-frequency” ultrasonic for frequencies over 0.8 MHz.

Systems for the generation of ultrasonic waves to treat biological tissues usually include a signal generator to generate the periodic electrical signals. Such systems have an ultrasonic head connected to the output of the signal generator through an electric circuit. The ultrasonic head often has a special surface for contacting the biological tissue. The periodic electrical signals created by the signal generator are transformed into ultrasonic waves in the area near the surface of the ultrasonic head.

Typically the ultrasonic head is designed to transform electrical signals with some fixed frequency. However some ultrasonic heads are known which can transform electric signals with different frequencies into corresponding ultrasonic waves. In the past, for example, some ultrasonic systems generated ultrasonic waves at three different frequencies, providing three different penetration depths into biological tissue.

Penetration depths and the ultrasonic intensity in the body depend on the ultrasonic frequency. One characteristic of the penetration depth is the so-called half-depth, which refers to the distance at which the sound intensity decreases to 50% of the initial value because of absorption. The lower the frequency the higher the penetration depth. For typical human biological tissue the half-depth for a frequency of 3 MHz is about 1 cm and for a frequency of 1 MHz it is about 3 cm. The half-depth is a relative value independent of the absolute ultrasound intensity.

In the past, the half-depth was regulated through the applied ultrasonic frequency and intensity which are integral characteristics of the signal. However, in many applications, it would be desirable to have more influence on the microscopic massage effect of the ultrasonic treatment, including on the relative movement of small portions of the treated biological tissue.

### SUMMARY

A system and a method extend the influence of ultrasonic waves on the biological tissues. The system provides new massage effects to the tissue. The system controls the ultrasonic frequencies generated by an ultrasound head to produce the massage effects.

The system switches between multiple different ultrasonic frequencies. The switching (modulation) frequency may be in the range between 10 Hz and 10,000 Hz, between 50 Hz and 5,000 Hz, or between 100 Hz and 2,000 Hz. Other frequency ranges may be used. The system produces a significantly more intensive massage effect in the tissue compared to the conventional systems.

The noted modulation frequencies optimally influence the elasticity of the tissue cells, and especially of the cell membranes. A much more intensive effect on the cells with a significant increase of the permeability of the cell membranes can be measured after switching at the above mentioned modulation frequencies. This improvement of the diffusion processes results in a distinct tightening of the tissue, which may be especially beneficial in the case of cellulite, but which may also be applied to the treatment of arthrosis, arthritis, and other conditions.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 shows a perspective view of an ultrasonic head.

FIG. 2 shows a block diagram of a system for the creation of ultrasonic waves.

FIG. 3 shows the acts that the system may take to produce ultrasonic waves at multiple different frequencies.

### DETAILED DESCRIPTION

The system switches an ultrasonic head between output of multiple different ultrasonic frequencies. The switching (modulation) frequency may be in the range between 10 Hz and 10,000 Hz, between 50 Hz and 5,000 Hz, or between 100 Hz and 2,000 Hz. Other frequency ranges may be used. The ultrasonic waves alternatively produce in the tissue regions of overpressure and underpressure. The pressure value is frequency independent. In particular, the pressure difference between the regions experiencing overpressure and those experiencing underpressure is frequency independent.

However, the distance between the regions experiencing overpressure and underpressure in the wave is frequency dependent. Regions experiencing overpressure are separated by the wavelength of the ultrasonic waves that the ultrasonic head is producing. The underpressure region is located approximately in the middle (at approximately the half wavelength position) of these two overpressure regions. The higher the frequency, the lower the wavelength and the smaller the distance between the regions with overpressure and underpressure in the wave. Although the absolute pressure value in the wave is frequency independent, the distance between the regions of overpressure and underpressure and so also the pressure gradient in the tissue will vary with the variation of the ultrasonic frequency.

The modulation frequency plays a significant role in the therapeutic effect of the treatment. The modulation frequency establishes how many times per second the ultrasonic head switches between the ultrasonic frequencies that the ultrasonic head produces.

The noted modulation frequencies optimally influence the elasticity of the tissue cells, and especially of the cell membranes. A much more intensive effect on the cells with a significant increase of the permeability of the cell membranes can be measured after switching at the above mentioned modulation frequencies. This improvement of the diffusion processes results in a distinct tightening of the tissue, which may be especially beneficial in the case of cellulite, but which may also be applied to the treatment of arthrosis, arthritis, and other conditions.

The normally applied frequencies many range from 0.7 MHz to approximately 10 MHz, but may include other frequency ranges in other implementations. Lower frequencies may used to obtain a higher penetration depth of a few centimeters. Higher frequencies may be used to obtain a penetration depth of less than 1 centimeter to treat superficial lesions or other conditions nearer the skin surface.

The frequency values between which the modulation switches may be selected to be relatively far apart from each other. For example, the frequencies may be selected to adhere to a ratio of between approximately 1:2 to 1:10 and particularly between approximately 1:2.5 to 1:5. The cells in the tissue are effected more intensively with these frequency relations.

In principle, the system may implement a selection of many different ultrasonic wave frequencies and modulation frequencies. In one implementation, the signal generator generates at least two frequencies which are transformed into ultrasonic waves of corresponding frequencies and the control unit regulates the modulation between the frequencies. In switching the frequencies, the system may modulate electrical signal frequency in the signal generator. Additionally or alternatively, the system may switch instead between the frequencies of the ultrasonic waves.

For switching the frequencies, the signal generator may include an oscillator and frequency modulation logic that creates at least two different frequencies. In this case the oscillator may generate a starting frequency. The frequency modulation logic produces the multiple desired frequencies from the starting frequency.

In one implementation, the signal generator includes an oscillator, a frequency controller and an amplifier. The oscillator creates the starting frequency and the oscillator frequency is regulated by the frequency controller which is connected to the oscillator. The oscillator output is connected to the input of the amplification unit. The amplified starting frequency signal will be produced at the output of the amplifier. The amplification factor may be set at the amplifier. The output of the amplifier is connected to the output of the signal generator so that the amplified signal can be transferred to the ultrasonic head.

In another implementation, a microcontroller implements the control unit. The control unit may be implemented using a computer or other programmable machine. The microcontroller is connected to the frequency controller so that the microcontroller sets the frequency produced at the signal output of the signal generator.

Furthermore, the control unit additionally may regulate the intensity of the ultrasonic wave. To that end, the control unit may be connected to a control input of the amplification unit or may be connected over a signal input to a signal output of the amplification unit. Through the signal input of the control unit, the actual intensity of the signal at the output of the signal generator can be checked. Therefore, the amplification factor may be corrected and an intensity control signal (e.g., a gain control signal) may be correspondingly transferred to the control input of the amplification unit. As a result, at the signal output, the signal with the preset intensity will be produced.

Furthermore, the ultrasonic wave generation system may employ multiple ultrasonic heads of different sizes. The ultrasonic heads of different sizes may be used for different application areas to contact the biological tissues. Depending on the form of the tissue and on the desired treatment area, a suitable ultrasonic head can be chosen. Accordingly, the signal output of the signal generator is provided to drive one or more of the multiple ultrasonic heads (e.g., multiple ultrasonic heads may be simultaneously connected to the signal output). In other words, one suitable ultrasonic head may be chosen for the application or several ultrasonic heads may be connected so that several areas can be treated with ultrasound simultaneously.

The system may include multiple ultrasonic heads that may be connected to the signal output of the signal generator. A signal control unit provides switching logic to automatically switch the signal to any of the ultrasonic heads. In one implementation, the signal control unit can store treatment programs and treatment parameters in a memory. The treatment parameters may establish, as examples, the treatment time, signal intensity, ultrasonic signal frequencies, and the modulation frequencies. The modulation frequencies may differ between the ultrasonic heads. The signal control unit may be connected to a control unit of the ultrasonic heads to establish, modify, add, or delete the treatment parameters and/or treatment programs. At the end of the preset treatment time the signal control unit may automatically switch to the next ultrasonic head specified in the program. The system may also include an output that indicates the current program, ultrasonic head, or any treatment parameter through an optical, acoustical, or other type of signal. The signal control unit transfers the parameters and programs to the control unit. The treatment can then be continued with the active ultrasonic head with the parameters and programs.

One particularly beneficial application of the system is use in combination with cosmetic liposuction. In part, the benefits arise due to the significant improvement of the permeability of the cell membranes through the ultrasonic treatment provided by the system. After application for liposuction, the fat can be suctioned or otherwise removed more easily and from larger areas than without pre-treatment with the system. At the same time, the pre-treatment causes a more homogeneous fat distribution. As a result, the typical irregularities of the skin after liposuction are greatly reduced.

The ultrasonic treatment may be applied before liposuction (e.g., once immediately prior to liposuction) and after liposuction (e.g., three to four times after the liposuction). The ultrasonic treatment may be performed each day or every second day or at other intervals. The treatment time may be between 30 and 60 minutes, but may vary widely.

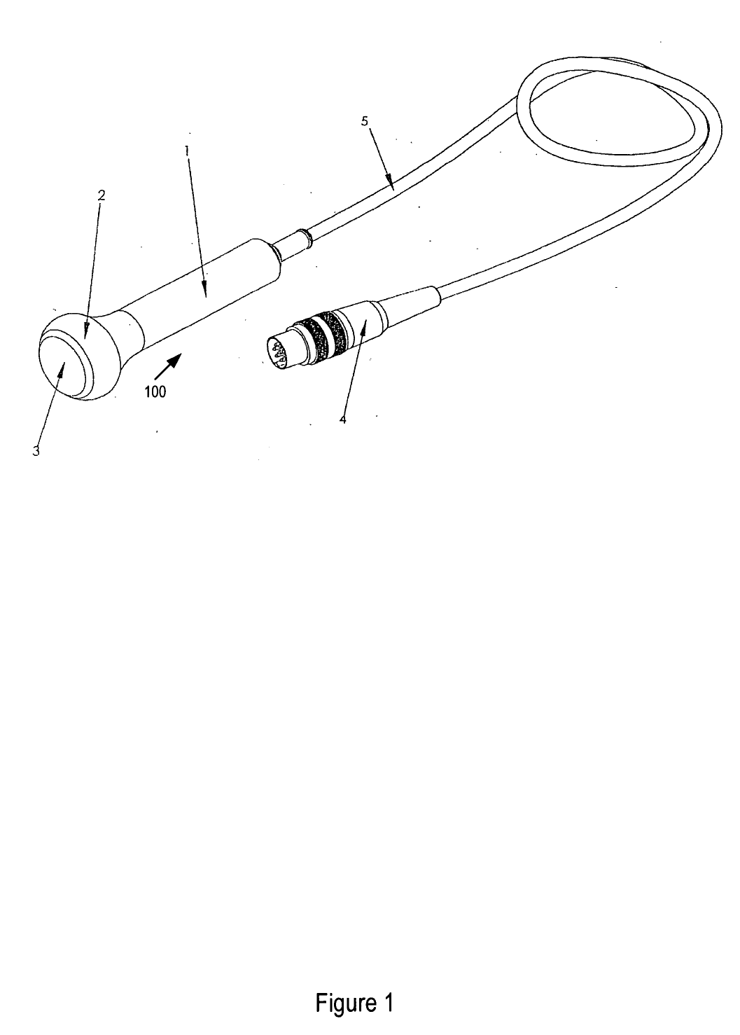

FIG. 1 shows an ultrasonic head 100 that includes a housing 1 with a head part 2 at the front where a protector 3 is arranged. At the outside of protector 3 there is a surface to contact the biological tissue. The protector 3 contacts from inside the piezoelectric element which creates the ultrasonic vibrations and transfers them to the protector 3. The housing 1 further contains corresponding microelectronics to control the piezoelectric element which is connected over a connector cable 5 and a plug connector 4 to a signal generator. The microelectronics transform, together with the piezoelectric element, the electrical signals which are transferred through the cable 5 into ultrasonic waves so that biological tissue can be treated with ultrasound waves by contacting the ultrasonic head 100.

The housing 1 further contains a coupling control 2. The coupling control 2 registers whether the protector 3 is in contact with the biological tissue. In this case the coupling control 2 gives a corresponding signal through the cable 5 and the plug connector 4 to the microcontroller of the system for the generation of ultrasonic waves.

The plug connector 4 may include a memory module that stores the calibration data of the ultrasonic head, for example the characteristic data of the piezoelectric element. The memory module may be connected to the microcontroller through the contacts in plug connector 4, so that the microcontroller may read the stored data and according to the calibration data select the intensity of the signals at the output of the signal generator in such a way that an ultrasonic radiation with a preset intensity will be generated by the piezoelectric element.

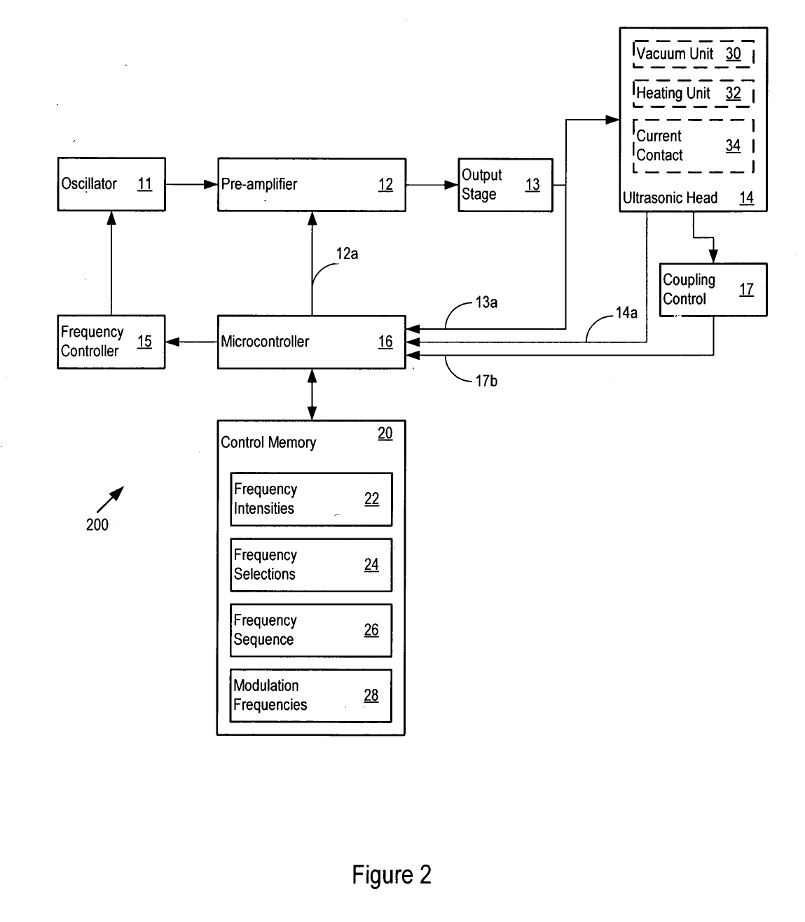

FIG. 2 shows a block diagram of the system 200 for the generation of ultrasonic radiation. An oscillator 11 generates periodic electrical signals which are then amplified in an amplification unit. This amplification unit includes a pre-amplifier 12 and an output stage 13. The signal from the output stage 13 is transferred to the ultrasonic head 14 through the plug connector 4 and the cable 5 shown in FIG. 1, so that the electrical signal may be transformed into ultrasonic waves in the ultrasonic head 14.

The frequency controller 15 regulates the frequency generated by the oscillator 11. A control unit 16 controls the frequency controller 15. For example, the control unit 16 may be a microcontroller that sets the currently selected frequency. The control unit 16 may include a control memory 20 that stores treatment parameters that direct the operation of the control unit 16. The treatment parameters include, as examples:

the desired intensities 22 of the ultrasonic radiation at one or more frequencies;

the ultrasonic frequencies 24 between the system 200 will switch;

the frequency sequence 26 through which the ultrasonic frequencies 24 are to be switched; and

the modulation frequency or frequencies 28 at which the system 200 switches the ultrasonic frequencies 24 according to the frequency sequence 26.

For example, assume that there are three ultrasonic frequencies: 1 MHz, 3 MHz and 10 MHz and that an intensity of 1 W/cm² and a modulation frequency of 100 Hz are set. The frequency sequence is 1 MHz, 3 MHz, 10 MHz, 3 MHz, 1 MHz, etc. The control unit 16, for 0.01 sec, provides a control signal to the frequency controller 15 for the generation of a frequency of 1 MHz; then, for 0.01 sec, a control signal for the generation of a frequency of 3 MHz; then, for 0.01 sec, a control signal for the generation of a frequency of 10 MHz. The control unit 16 then repeats the sequence until treatment ends.

Accordingly, a corresponding periodic signal with the desired frequency will be created and can be found at the output of the output stage 13. This output is connected through a feedback connection 13a to a signal input of the control unit 16. The control unit 16 may monitor the actual signal intensity at the output stage 13. In response, the control unit 16, may, for example, provide a pre-amplification control signal through the connection 12a to the pre-amplifier 12 in order to provide a signal with the desired intensity at the output of the output stage 13. To calculate the desired intensity of the electrical signal at the output of the output stage 13, the control unit 16 may read the calibration data for the ultrasonic head 14 (e.g., from the memory module in the plug connector 4) through the connection 14a. The control unit 16 calculates, based in part on the calibration data, the desired intensity at the output of the output stage 13 and controls the pre-amplifier 12 so that the ultrasonic head generates ultrasonic radiation with the desired intensity of 1 W/cm².

Furthermore, the control unit 16 may be connected through the cable 17b to the coupling control 7 of the ultrasonic head 4. When the protector 3 (shown in FIG. 1) is in contact with the biological tissue, the coupling control asserts a contact signal to the control unit 16. The control unit 16 may be operable to enable the pre-amplifier when there is a signal at the coupling control 17 and disable the pre-amplifier when there is no signal at the coupling control 17. Accordingly, when there is no signal at the coupling control 17 (i.e., when the protector 3 shown in FIG. 1 does not contact the biological tissue), the control unit 16 disables the pre-amplifier 12. As a result, there is only a low-intensity signal at the output of the output stage 13.

The system 200 may also include a stimulation current generator. The stimulation current generator provides a stimulation current in combination with the ultrasound treatment of the biological tissues. The stimulation current output is connected to a contact unit 34, for example an electrode, so that the stimulation current may be transferred through this contact unit 34 to the biological tissue. The contact unit 34 may be integrated into the ultrasonic head 14. As a result, contacting the ultrasonic head 14 to the biological tissue provides both stimulation current and ultrasound delivery to the tissue.

The system 200 may also include a vacuum unit 30 with a vacuum connection. The vacuum unit 30 may be combined with the ultrasonic head 14. As a result, by application of the ultrasonic head 14 to the biological tissue, an underpressure will be simultaneously created. The vacuum unit 30 may, for example, be designed as a vacuum bell. The vacuum bell forms a structural unit with the ultrasonic head 14, so that by contacting the biological tissue a roughly fluid-tight connection will be established.

The system 200 may also include a heating unit 32 for treating biological tissue. The heating unit 32, for example, may be an infrared heating unit. The infrared heating unit may include an infrared radiation source, such as a glow tube, and an energy supply for the infrared radiation source. The infrared radiation source may be integrated into the ultrasonic head 14 and disposed such that the contact between the ultrasonic head 14 and the tissue will also provide the infrared irradiation to the tissue.

The system may also be implemented with current generation logic operable to generate high frequency currents. The high frequency currents, for example between 300 kHz and 10 MHz, may be transferred to the tissue through one or more external electrodes. The high frequency current may be applied simultaneously with, or before or after the ultrasonic treatment.

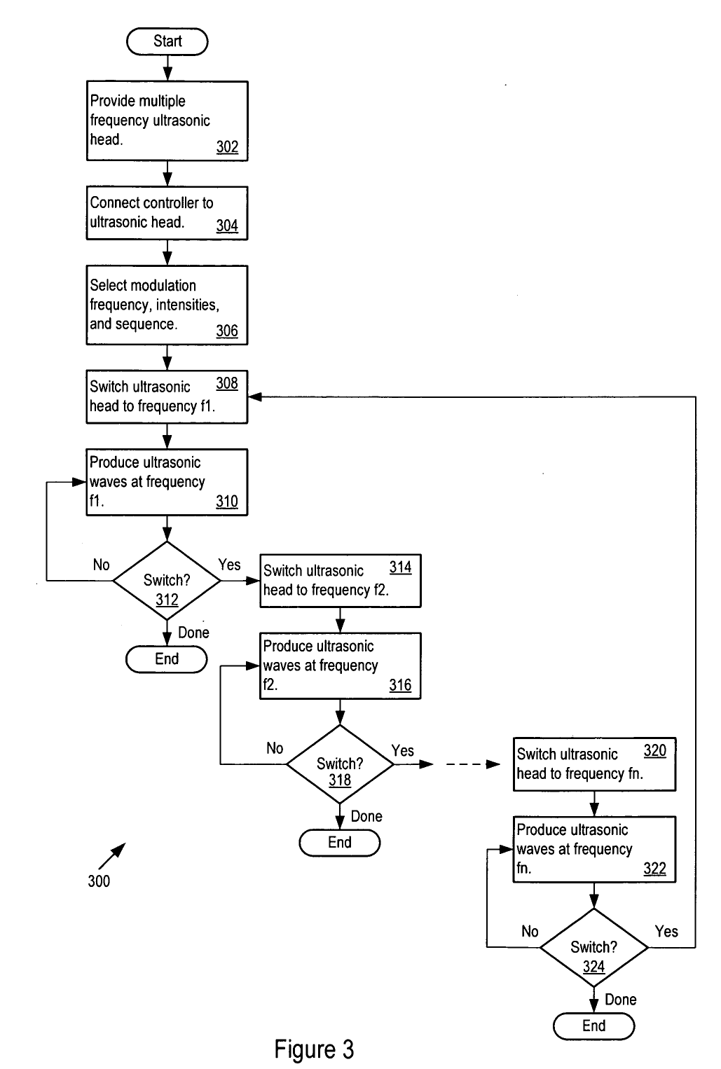

FIG. 3 shows the acts 300 that the system 200 may take to generate and switch between multiple ultrasonic frequencies. The system 200 provides a multiple frequency ultrasonic head, or multiple ultrasonic heads operable to generate one or more ultrasonic frequencies (Act 302). The ultrasonic heads are connected to the output stage 13 and control unit 16 (Act 304). The control unit 16 determines (e.g., reads from the control memory 20), the frequency intensities 22, frequency selections 24, frequency sequence 26, and the modulation frequency 28 parameters (Act 306).

The system 200 then switches to the first ultrasonic frequency, f1, in the frequency sequence (Act 308). The control unit 16 generates the frequency control and amplification control signals to produce the first ultrasonic frequency, f1, at the specified intensity (Act 310). The control unit 16 determines whether the modulation time interval (e.g., 0.01 sec for a 100 Hz modulation frequency) has passed for switching to the next frequency, f2, in the frequency sequence (Act 312). If it is not yet time to switch, the system 200 continues to generate the first ultrasonic frequency, f1.

When it is time to switch, the control unit 16 switches to the next ultrasonic frequency, f2, in the frequency sequence. Accordingly, the control unit 16 generates frequency control and amplification control signals to produce the second ultrasonic frequency, f2, at the specified intensity (Act 316). The system 200 again determines whether it is time to switch to a new frequency (Act 318). The treatment process proceeds to each frequency specified in the frequency sequence. At the last frequency, the system 200 switches to the last ultrasonic frequency, fn, in the frequency sequence (Act 320). The control unit 16 generates frequency control and amplification control signals to produce the last ultrasonic frequency, fn, at the specified intensity (Act 322).

The control unit 16 determines when the modulation time interval has passed (Act 324). After the last ultrasonic frequency, fn, in the frequency sequence, the control unit 16 begins again at the first ultrasonic frequency (Act 308). If at any time the treatment ends, the system 200 may stop producing the ultrasonic signals, heat output, vacuum output, stimulation current, or other treatment feature.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

What is claimed is:

1. A system for generating ultrasonic waves, the system comprising:
an ultrasonic head operable to produce ultrasonic waves at a first frequency f1 and ultrasonic waves at a second frequency f2;
a control unit operable to switch the ultrasonic head between production of the ultrasonic waves at the first frequency f1 and production of the ultrasonic waves at the second frequency f2; and
where the control unit is operable to switch the ultrasonic head at a modulation frequency between approximately 10 Hz and 10,000 Hz, between approximately 50 Hz and 5,000 Hz, or approximately 100 Hz and 2,000 Hz.

2. The system according to claim 1, characterized in that the ultrasonic waves have the frequencies in a range between 20 kHz and 20 MHz, in particular between 0.5 MHz and approx. 10 MHz, preferably between 0.7 MHz and approx. 10 MHz.

3. The system according to claim 1, characterized in that the switching takes place between the ultrasonic waves with frequencies of an approximate ratio of 1:2 to 1:10, in particular of 1:2.5 to 1:5, preferably of approx. 1:3 to each other.

4. The system according to claim 1, characterized in that there is a signal generator which generates at least two different frequencies f1, f2 which are transformed into ultrasonic waves with corresponding frequencies.

5. The system according to claim 1, characterized in that the signal generator has an oscillator and a frequency modulation unit which creates at least two different frequencies from the oscillator frequency.

6. The system according to claim 4, characterized in that the signal generator has an oscillator, a frequency controller and an amplification unit; the frequency controller is connected to the oscillator and so designed that for a preset frequency it has such an effect on the oscillator that it can oscillate with the preset frequency; the output of the oscillator is connected to the input of the amplification unit and the amplification unit is so designed that it amplifies the signal at the input according to a preset factor and that the output of the amplification unit is at the same time the signal output of the signal generator.

7. The system according to claim 6, characterized in that the control unit is designed as a microcontroller with a control output which is connected to the input of the frequency controller to control the frequency created by the signal generator.

8. The system according to claim 7, characterized in that the control unit is connected to a control input of the amplification unit that the control unit has a signal input which is connected to the signal output of the amplification unit and that the control unit is so designed that dependent on the signal intensity at the signal input of the control unit it has such an effect on the control input of the amplification unit that the amplification unit generates a periodic electrical signal with an intensity preset through the control unit.

9. The system according to claim 1, characterized in that the system has at least two ultrasonic heads especially of different sizes and that the signal output of the signal generator is so designed that optionally one or at least two ultrasonic heads can be connected simultaneously to the signal output of the signal generator.

10. The system according to claim 1, characterized in that the system has in addition a stimulation current generator with a stimulation current output and that the ultrasonic head has a contact unit for the transmission of a stimulation current to the biological tissue and is so designed, that the contact unit can be connected to the stimulation current output of the stimulation current generator.

11. The system according to claim 1, characterized in that the system has in addition a vacuum unit with a vacuum connection for the creation of the underpressure and that the ultrasonic head has a vacuum bell and is so designed that the vacuum bell can be connected to the vacuum unit; the ultrasonic head and the vacuum bell are so designed that by contact of the ultrasonic head to the biological tissue the vacuum bell has almost a fluid-tight contact to the biological tissue so that there can be created a underpressure between the biological tissue and the ultrasonic head.

12. The system according to claim 1, characterized in that the system has in addition an infrared unit to generate the infrared radiation with at least one infrared radiation source for the generation of infrared radiation and an energy supply which is connected with the infrared radiation source; the infrared radiation source forms together with the ultrasonic head one structural unit.

13. The system according to claim 1, characterized in that the system in addition has a unit for the generation of high frequency currents with frequencies between 300 kHz and 10 MHz and that this current can be transferred to at least one external electrode.

14. A method for the generation of ultrasonic waves, comprising:
providing an electrically controlled ultrasonic head operable to generate ultrasonic waves at multiple different frequencies; and
switching between the multiple different frequencies at a modulation frequency between approximately 10 Hz and 10,000 Hz, between approximately 50 Hz and 5,000 Hz, or between approximately 100 Hz and 2,000 Hz.

15. A method for the cosmetic treatment of adipose tissue particularly by selective liposuction of certain body areas, comprising:
determining the body areas to be treated with liposuction;
before or after the liposuction:
providing an electrically controlled ultrasonic head operable to generate ultrasonic waves at multiple different frequencies;
positioning the electrically controlled ultrasonic head adjacent the body areas; and
switching between the multiple different frequencies at a modulation frequency between approximately 10 Hz and 10,000 Hz, between approximately 50 Hz and 5,000 Hz, or between approximately 100 Hz and 2,000 Hz.
